(19) United States
(12) Patent Application Publication
(10) Pub. No.: US 2013/0064891 A1
(43) Pub. Date: Mar. 14, 2013

(54) PHARMACEUTICAL COMPOSITIONS OF NSAID AND ACID INHIBITOR

(71) Applicants: Ashok Sahoo, Pune (IN); Ashish Guha, Pune (IN); Shrenik Kole, Pune (IN); Makrand Avachat, Pune (IN)

(72) Inventors: Ashok Sahoo, Pune (IN); Ashish Guha, Pune (IN); Shrenik Kole, Pune (IN); Makrand Avachat, Pune (IN)

(21) Appl. No.: 13/699,047

(22) PCT Filed: May 20, 2011
(86) PCT No.: PCT/IB11/01085

§ 371 Date: Nov. 20, 2012

(30) Foreign Application Priority Data
May 21, 2010 (IN) 560/KOL/2010

(51) Int. Cl. A61K 31/4439; A61K 9/20; A61P 1/04; A61P 29/00; A61P 19/02; A61K 9/00; A61K 31/616

(52) U.S. Cl. 424/465; 514/338; 424/400; 514/161

(57) ABSTRACT

The present invention relates a pharmaceutical composition comprising esomeprazole magnesium dihydrate, non-steroidal anti-inflammatory drug and one or more pharmaceutically acceptable excipients, wherein non-steroidal anti-inflammatory drug is present in two or more portions. More particularly, the invention relates to compositions comprising Esomeprazole and Naproxen and a process for preparation thereof. Further invention relates to a pharmaceutical composition comprising esomeprazole magnesium dihydrate, non-steroidal anti-inflammatory drug and one or more pharmaceutically acceptable excipients wherein, esomeprazole magnesium dihydrate and non-steroidal anti-inflammatory drug provides co-ordinated release.

### FIELD OF THE INVENTION

The present invention relates to a pharmaceutical composition which comprises an acid inhibitor in combination with one or more NSAID(s), the process of preparing such compositions and their use in medicine.

### BACKGROUND OF THE INVENTION

NSAID(s) are among the most commonly prescribed and used drugs world-wide. Despite the therapeutic benefits of NSAID(s), their use is frequently limited by an increased risk of gastrointestinal side-effects, mainly upper gastrointestinal side-effects like peptic ulceration, dyspeptic symptoms gastroduodenal lesions, e.g., ulcers and erosions, in susceptible individuals.

Attempts to modify the NSAID(s) structure in order to prevent such side-effects have so far been less successful. The most promising solution to the problem of healing and preventing NSAID associated upper gastrointestinal problems like ulcers and dyspeptic symptoms in patients with a need for continuous NSAID(s) treatment is to combine the NSAID(s) treatment with an acid inhibitor such as prostaglandin analogues, H₂-receptor antagonists or proton pump inhibitors for the healing and/or prophylaxis of NSAID(s) associated gastrointestinal side-effects.

Some of the patents which discloses most promising solution to the problem of healing and preventing NSAID associated upper gastrointestinal problems are as follows

U.S. Pat. No. 5,204,118; U.S. Pat. No. 5,417,980 have disclosed pharmaceutical compositions for treating the symptoms of overindulgence with a combination of non-steroidal anti-inflammatory drug or acetaminophen and a histamine receptor blocker and/or a proton pump inhibitor composition.

U.S. Pat. No. 6,613,354 discloses capsule formulation comprising an acid susceptible proton pump inhibitor, one or more Non Steroidal Antiinflammatory Drugs (NSAID(s)), an enteric coating layer to protect the proton pump inhibitor and, optionally, pharmaceutically acceptable excipients.

U.S. Pat. No. 5,601,843 discloses pharmaceutical composition including a core of an NSAID selected from diclofenac and piroxicam which core is further surrounded by a mantle coating of a prostaglandin, wherein an intermediate coating can be present between the NSAID core and prostaglandin mantle coating.

U.S. Pat. No. 5,466,436 discloses co-administration of a non-steroidal anti-inflammatory drug with a salt formed between ranitidine and a complex of bismuth with a carboxylic acid.

U.S. Pat. No. 5,037,815 discloses combination of NSAID(s) and acid inhibitors like proton pump inhibitor, H₁ or H₂ receptor blocker.

U.S. Pat. No. 6,926,907 (US '907) directed to pharmaceutical compositions that provide for the co-ordinated release of an acid inhibitor and a non-steroidal anti-inflammatory drug (NSAID). US '907 patent discloses a unit dosage form which provides coordinated release such that NSAID is surrounded by a coating that prevents the release of essentially any NSAID unless the pH of the surrounding medium is 3.5 or higher; further '907 patent also discloses that at least a portion of acid inhibitor is not surrounded by an enteric coating and released regardless of whether the pH of the surrounding medium is below 3.5 or above 3.5.

While there are many compositions available to reduce the gastrointestinal side effects of NSAID(s), still there remains a need to develop a composition of NSAID(s) in combination with acid inhibitor.

### OBJECTIVE OF THE INVENTION

The main objective of the invention is to provide pharmaceutical compositions comprising a combination of therapeutically effective amount of one or more non-steroidal anti-inflammatory drug (NSAIDs) and acid inhibitor wherein the acid inhibitor is capable of raising the pH of the GI tract of patients.

Another object of the invention is a pharmaceutical composition comprising Esomeprazole magnesium dihydrate, non-steroidal anti-inflammatory drug and one or more pharmaceutically acceptable excipients.

Another object of the invention is a pharmaceutical composition comprising Esomeprazole magnesium dihydrate, non-steroidal anti-inflammatory drug and one or more pharmaceutically acceptable excipients wherein, Esomeprazole magnesium dihydrate and non-steroidal anti-inflammatory drug provides co-ordinated release.

Another object of the invention is a pharmaceutical composition comprising Esomeprazole magnesium dihydrate, non-steroidal anti-inflammatory drug and one or more pharmaceutically acceptable excipients, wherein non-steroidal anti-inflammatory drug is present in two or more portions.

Another object of the invention is a pharmaceutical composition comprising Esomeprazole magnesium dihydrate, non-steroidal anti-inflammatory drug and one or more pharmaceutically acceptable excipients, wherein two or more portions of non-steroidal anti-inflammatory drug are separated by barrier layer.

Another object of the invention is a pharmaceutical composition comprising Esomeprazole magnesium dihydrate, non-steroidal anti-inflammatory drug and one or more pharmaceutically acceptable excipients, wherein non-steroidal anti-inflammatory drug is present in two or more portions, such that esomeprazole magnesium dihydrate is in immediate release portion and non-steroidal anti-inflammatory drug is in two portions, a delayed release portion and an immediate release portion.

### SUMMARY OF THE INVENTION

The present invention provides pharmaceutical compositions comprising a combination of therapeutically effective amount of one or more NSAIDs and acid inhibitor and the process of preparing such compositions and their use in medicine. The present invention relates a pharmaceutical composition comprising Esomeprazole magnesium dihydrate, non-steroidal anti-inflammatory drug (NSAID) and one or more pharmaceutically acceptable excipients, wherein non-steroidal anti-inflammatory drug is present in two or more portions. More particularly, the invention relates to compositions comprising esomeprazole magnesium dihydrate and NSAID providing co-ordinated release. and a process for preparation thereof.

### DETAILED DESCRIPTION OF THE INVENTION

The present invention is directed towards pharmaceutical compositions comprising a combination of therapeutically effective amount of one or more NSAIDs and acid inhibitor wherein the acid inhibitor is capable of raising the pH of the Gastro-intestinal (GI) tract of patients.

In preferred embodiment, a pharmaceutical composition is unit dosage form suitable for oral administration to a patient. The term “unit dosage form” as used herein refers to a single entity for drug administration. For example, a single tablet or capsule combining both an acid inhibitor and an NSAID would be a unit dosage form.

The term “acid inhibitor” refers to agent(s) that inhibit gastric acid secretion and increase gastric pH. The acid inhibitor according to the present invention comprises but not limited to H2 blocker and proton pump inhibitor the examples of which are but not limited to cimetidine, ranitidine, ebrotidine, pabutidine, lafutidine, loxtidine, famotidine, omeprazole, esomeprazole, pantoprazole, lansoprazole, dexlansoprazole, rabeprazole, leminoprazole etc.

The “NSAIDs” comprises but not limited to COX-2 inhibitor such as celecoxib, rofecoxib, meloxicam, piroxicam, valdecoxib, parecoxib, etoricoxib, CS-502, JTE-522, L-745,337 or NS398. Alternatively, the NSAID may be aspirin, acetaminophen, ibuprofen, flurbiprofen, ketoprofen, naproxen, oxaprozin, etodolac, indomethacin, ketorolac, lornoxicam, nabumetone, or diclofenac etc.

NSAIDs can be used interchangeably with non-steroidal anti-inflammatory drug.

It will be understood that, for the purposes of the present invention acid inhibitor and NSAIDs will include all common forms of these compounds and, in particular, their pharmaceutically acceptable salt(s) or enantiomer(s) or polymorph(s) thereof.

In one embodiment, acid inhibitor present in the pharmaceutical composition of invention is esomeprazole magnesium dihydrate or bis(5-methoxy-2-[(S)-[(4-methoxy-3,5-dimethyl-2-pyridinyl)methyl]sulfinyl]-1H-benzimidazole-1-yl) magnesium dihydrate characterised by the X-ray powder diffractogram as disclosed in FIG. 2.

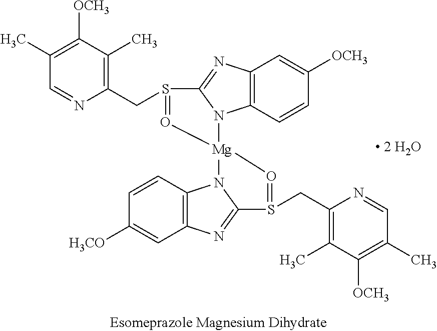

Esomeprazole magnesium dihydrate present in the pharmaceutical composition of invention is in amorphous form, crystalline form or mixture thereof.

A ‘Therapeutically effective amounts’ of NSAIDs and acid inhibitor mentioned in the present application are those in common practice and known to person skilled in the art.

A ‘Therapeutically effective amount’ of NSAIDs comprises but is not limited to an amount effective to reduce or eliminate pain or inflammation which is safe and well tolerated in patients with acceptable adverse effect profiles.

A ‘Therapeutically effective amount’ of acid inhibitor comprises but is not limited to an amount capable of raising the pH of the GI tract of patients.

A pharmaceutical composition according to the present invention comprises but is not limited to tablets (single layered tablets, multilayered tablets, mini tablets, bioadhesive tablets, caplets, matrix tablets, tablet within a tablet, mucoadhesive tablets, modified release tablets, pulsatile release tablets, and timed release tablets), pellets, beads, granules, sustained release formulations, capsules, microcapsules, tablets in capsules, microspheres, matrix formulations, microencapsulation.

In another embodiment, a pharmaceutical composition comprising esomeprazole magnesium dihydrate, non-steroidal anti-inflammatory drug and one or more pharmaceutically acceptable excipients.

In another embodiment, a pharmaceutical composition comprising esomeprazole magnesium dihydrate, non-steroidal anti-inflammatory drug and one or more pharmaceutically acceptable excipients, wherein esomeprazole magnesium dihydrate and non-steroidal anti-inflammatory drug provides co-ordinated release.

In another embodiment, a multi-layered tablet composition comprising esomeprazole magnesium dihydrate, non-steroidal anti-inflammatory drug and one or more pharmaceutically acceptable excipients, wherein esomeprazole magnesium dihydrate and non-steroidal anti-inflammatory drug provides co-ordinated release.

In another embodiment, a capsule composition comprising esomeprazole magnesium dihydrate, non-steroidal anti-inflammatory drug and one or more pharmaceutically acceptable excipients, wherein esomeprazole magnesium dihydrate and non-steroidal anti-inflammatory drug provides co-ordinated release.

The term “co-ordinated release” refers to release such that, the non-steroidal anti-inflammatory drug is surrounded by a coating that, upon ingestion of a pharmaceutical composition by the patient, prevents the release of essentially any NSAID from said dosage form unless the pH of the surrounding medium is 3.5 or higher; ii) at least a portion of said acid inhibitor is not surrounded by an enteric coating and, upon ingestion of said pharmaceutical composition by said patient, is released regardless of whether the pH of the surrounding medium is below 3.5 or above 3.5.

In another embodiment, a pharmaceutical composition comprises a combination of one or more NSAID(s) and acid inhibitor wherein the NSAID(s) comprising two portions, a delayed release portion and an immediate release portion wherein NSAID(s) present in a delayed release portion and an immediate release portion are same or different and an acid inhibitor is present in immediate release portion.

The delayed release portion of a pharmaceutical composition of invention releases NSAID from pharmaceutical composition at a slower rate than that from an immediate release portion.

In another embodiment, a pharmaceutical composition comprises combination of one or more NSAID(s) and acid inhibitor; wherein the NSAID(s) comprises two portions, a delayed release portion and an immediate release portion, and an acid inhibitor is present in immediate release portion; wherein immediate release portion of NSAID(s) and immediate release portion of an acid inhibitor are same or different.

In another embodiment, a pharmaceutical composition comprises a combination of one or more NSAID(s) and proton pump inhibitor; wherein the NSAID(s) comprises two portions, a delayed release portion and an immediate release portion; wherein NSAID(s) present in a delayed release portion and an immediate release portion are same or different and proton pump inhibitor is present in immediate release portion.

In another embodiment, a pharmaceutical composition comprising esomeprazole magnesium dihydrate, non-steroidal anti-inflammatory drug and one or more pharmaceutically acceptable excipients, wherein non-steroidal anti-inflammatory drug is present in two or more portions.

In another embodiment, a pharmaceutical composition comprises a combination of one or more NSAID(s) and H₂ blocker; wherein the NSAID(s) comprises two portions, a delayed release portion and an immediate release portion wherein NSAID(s) present in a delayed release portion and an immediate release portion are same or different and H₂ blocker is present in immediate release portion.

In another embodiment, a pharmaceutical composition comprises a combination of NSAID selected from naproxen or aspirin and acid inhibitor selected from omeprazole or esomeprazole or famotidine, wherein the naproxen or aspirin comprises two portions, a delayed release portion and an immediate release portion and omeprazole or esomeprazole or famotidine is present in immediate release portion.

In another embodiment, a pharmaceutical composition comprising esomeprazole magnesium dihydrate, non-steroidal anti-inflammatory drug and one or more pharmaceutically acceptable excipients, wherein two or more portions of non-steroidal anti-inflammatory drug are separated by barrier layer.

In another embodiment, a pharmaceutical composition is a capsule or tablet comprising a combination of one or more NSAID(s) and an acid inhibitor, wherein a delayed release portion of NSAID(s) comprising enteric coated pellets and an immediate release portion comprises NSAID(s) and acid inhibitor.

In another embodiment, a pharmaceutical composition is a capsule or tablet comprising a combination of one or more NSAID(s) and proton pump inhibitor, wherein a delayed release portion of NSAID(s) comprising enteric coated pellets and an immediate release portion comprises NSAID(s) and proton pump inhibitor.

In another embodiment, a pharmaceutical composition comprising esomeprazole magnesium dihydrate, non-steroidal anti-inflammatory drug and one or more pharmaceutically acceptable excipients, wherein non-steroidal anti-inflammatory drug is present in two or more portions, such that esomeprazole magnesium dihydrate is in immediate release portion and non-steroidal anti-inflammatory drug is in two portions, a delayed release portion and an immediate release portion.

In another embodiment, a pharmaceutical composition is a capsule or tablet comprising a combination of one or more NSAID(s) and H₂ blocker; wherein a delayed release portion of NSAID(s) comprising enteric coated pellets and an immediate release portion comprises NSAID(s) and H₂ blocker.

In another embodiment, a pharmaceutical composition is a capsule or tablet comprising a combination of NSAID selected from naproxen or aspirin and acid inhibitor selected from omeprazole or esomeprazole or famotidine, wherein a delayed release portion of naproxen or aspirin comprising enteric coated pellets and an immediate release portion comprises naproxen or aspirin and omeprazole or esomeprazole or famotidine.

In another embodiment, a pharmaceutical composition is a capsule comprising a combination of one or more NSAID(s) and an acid inhibitor; wherein a delayed release portion of NSAID(s) comprising enteric coated pellets or minitablets, wherein an immediate release portion is a bilayer tablet comprising NSAID(s) and an acid inhibitor.

In another embodiment, a pharmaceutical composition is a capsule comprising a combination of one or more NSAID(s) and proton pump inhibitor; wherein a delayed release portion of NSAID(s) comprising enteric coated pellets or minitablets; wherein an immediate release portion is a bilayer tablet comprising NSAID(s) and proton pump inhibitor.

In another embodiment, a pharmaceutical composition is a capsule comprising a combination of one or more NSAID(s) and H₂ blocker; wherein a delayed release portion of NSAID(s) comprising enteric coated pellets or minitablets; wherein an immediate release portion is a bilayer tablet comprising NSAID(s) and of H₂ blocker.

In another embodiment, a pharmaceutical composition is a capsule comprising a combination of NSAID selected from naproxen or aspirin and acid inhibitor selected from omeprazole or esomeprazole or famotidine; wherein a delayed release portion of naproxen or aspirin comprising enteric coated pellets or minitablets; wherein an immediate release portion is a bilayer tablet comprising naproxen or aspirin and omeprazole or esomeprazole or famotidine.

In another embodiment, a pharmaceutical composition is a capsule comprising a combination of one or more NSAID(s) and an acid inhibitor; wherein a delayed release portion of NSAID(s) comprising enteric coated pellets or minitablets; wherein an immediate release portion comprising simple mixture of NSAID(s) and an acid inhibitor.

In another embodiment, a pharmaceutical composition is a capsule comprising a combination of one or more NSAID(s) and proton pump inhibitor; wherein a delayed release portion of NSAID(s) comprising enteric coated pellets or minitablets; wherein an immediate release portion comprising simple mixture of NSAID(s) and an proton pump inhibitor.

In another embodiment, a pharmaceutical composition is a capsule comprising a combination of one or more NSAID(s) and H₂ blocker; wherein a delayed release portion of NSAID(s) comprising enteric coated pellets or minitablets; wherein an immediate release portion comprising simple mixture of NSAID(s) and an H₂ blocker.

In another embodiment, a pharmaceutical composition is a capsule comprising a combination of NSAID selected from naproxen or aspirin and omeprazole or acid inhibitor selected from esomeprazole or famotidine; wherein a delayed release portion of naproxen or aspirin comprising enteric coated pellets or minitablets; wherein an immediate release portion comprising simple mixture of naproxen or aspirin and an omeprazole or esomeprazole or famotidine.

In another embodiment, a pharmaceutical composition is an inlayed tablet or tablet in tablet comprising a combination of one or more NSAID(s) and an acid inhibitor; wherein a delayed release portion of NSAID(s) comprising enteric coated tablet; wherein an immediate release portion is combination of NSAID(s) and an acid inhibitor.

In another embodiment, a pharmaceutical composition is an inlayed tablet or tablet in tablet comprising a combination of one or more NSAID(s) and proton pump inhibitor; wherein a delayed release portion of NSAID(s) comprising enteric coated tablet; wherein an immediate release portion is combination of NSAID(s) and proton pump inhibitor.

In another embodiment, a pharmaceutical composition is an inlayed tablet or tablet in tablet comprising a combination of one or more NSAID(s) and H₂ blocker; wherein a delayed release portion of NSAID(s) comprising enteric coated tablet; wherein an immediate release portion is combination of NSAID(s) and H₂ blocker.

In another embodiment, a pharmaceutical composition is an inlayed tablet or tablet in tablet comprising a combination of NSAID selected from naproxen or aspirin and acid inhibitor selected from omeprazole or esomeprazole or famotidine; wherein a delayed release portion of naproxen or aspirin comprising enteric coated tablet; wherein an immediate release portion is combination of naproxen or aspirin and omeprazole or esomeprazole or famotidine.

In another embodiment, a pharmaceutical composition is multilayered tablet comprising a combination of one or more NSAID(s) and an acid inhibitor; wherein a delayed release portion of NSAID(s) comprises enteric coated pellets or granules; wherein an immediate release portion comprises NSAID(s) and an acid inhibitor.

In another embodiment, a pharmaceutical composition is multilayered tablet comprising a combination of one or more NSAID(s) and proton pump inhibitor; wherein a delayed release portion of NSAID(s) comprises enteric coated pellets or granules; wherein an immediate release portion comprises NSAID(s) and proton pump inhibitor.

In another embodiment, a pharmaceutical composition is multilayered tablet comprising a combination of one or more NSAID(s) and H₂ blocker; wherein a delayed release portion of NSAID(s) comprises enteric coated pellets or granules; wherein an immediate release portion comprises NSAID(s) and H₂ blocker.

In another embodiment, a pharmaceutical composition is multilayered tablet comprising a combination of NSAID selected from naproxen or aspirin and acid inhibitor selected from omeprazole or esomeprazole or famotidine; wherein a delayed release portion of naproxen or aspirin comprises enteric coated pellets or granules; wherein an immediate release portion comprises naproxen or aspirin and omeprazole or esomeprazole or famotidine.

A delayed release portion of pharmaceutical composition of invention comprises about −90% to about 99% of NSAID(s) and preferably about 94% to about 96% of NSAID(s).

An immediate release portion of pharmaceutical composition of invention comprises about 1% to about 10% of NSAID(s) preferably about 4% to about 6% of NSAID(s).

The delayed release and one or more immediate release portions according to the present invention comprises but not limited to granules, spheroids, microspheres, seeds, pellets, beads, microcapsules, agglomerates, minitablets or tablets that are manufactured by conventional techniques using pharmaceutically acceptable excipient(s), wherein delayed release and one or more immediate release portions are hydrophilic or hydrophobic.

The hydrophilic portion according to the present invention comprises but not limited to celluloses or their salts or derivatives thereof, hydroxyethylcellulose, hydroxypropyl cellulose, hydroxypropyl methylcellulose, sodium carboxymethyl cellulose, alginic acid or their salts and derivatives thereof, carbomer (Carbopol™), polyethyleneoxide, xanthan gum, guar gum, locust bean gum, poly vinyl acetate, polyvinyl alcohol, lactose, PVA these hydrophilic polymers also act as pore forming agent.

The hydrophobic portion according to the present invention comprises but not limited to Ammonio methacrylate copolymers type A and B as described in USP, methacrylic acid copolymer type A, B and C as described in USP, Polyacrylate dispersion 30% as described in Ph. Eur., Polyvinyl acetate dispersion, ethylcellulose, cellulose acetate, cellulose propionate (lower, medium or higher molecular weight), cellulose acetate propionate, cellulose acetate butyrate, cellulose acetate phthalate, cellulose triacetate, poly(methyl methacrylate), poly(ethyl methacrylate), poly(butyl methacrylate), poly(isobutyl methacrylate), and poly(hexyl methacrylate). Poly(isodecyl methacrylate), poly(lauryl methacrylate), poly(phenyl methacrylate), poly(methyl acrylate), poly(isopropyl acrylate), poly(isobutyl actylate), poly(octadecyl acrylate), waxes such as beeswax, carnauba wax, microcrystalline wax, and ozokerite; fatty alcohols such as cetostearyl alcohol, stearyl alcohol; cetyl alcohol and myristyl alcohol; and fatty acid esters such as glyceryl monostearate, glycerol distearate; glycerol monooleate, acetylated monoglycerides, tristearin, tripalmitin, cetyl esters wax, glyceryl palmitostearate, glyceryl behenate, and hydrogenated castor oil.

The term ‘pharmaceutically acceptable excipient(s)’ used in the pharmaceutical compositions of invention comprise but not limited to diluents, binders, pH stabilizing agents, disintegrants, surfactants, glidants and lubricants.

The amounts of excipient(s) employed will depend upon how much active agent is to be used. One excipient(s) can perform more than one function.

Binders as used in the present invention comprises but are not limited to, starches such as potato starch, wheat starch, corn starch; microcrystalline cellulose such as products known under the registered trade marks Avicel, Filtrak, Heweten or Pharmacel; celluloses such as hydroxypropyl cellulose, hydroxyethyl cellulose, hydroxypropylmethyl cellulose (HPMC), ethyl cellulose, sodium carboxy methyl cellulose; natural gums like acacia, alginic acid, guar gum; liquid glucose, dextrin, povidone, syrup, polyethylene oxide, polyvinyl pyrrolidone, poly-N-vinyl amide, polyethylene glycol, gelatin, poly propylene glycol, tragacanth, combinations there of and other materials known to one of ordinary skill in the art and mixtures thereof.

Fillers or diluents, as used in the present invention comprises but not limited to confectioner's sugar, compressible sugar, dextrates, dextrin, dextrose, fructose, lactitol, mannitol, sucrose, starch, lactose, xylitol, sorbitol, talc, microcrystalline cellulose, calcium carbonate, calcium phosphate dibasic or tribasic, calcium sulphate, and the like can be used.

Lubricants as used in the present invention comprises but not limited to Mg, Al, Ca or Zn stearate, polyethylene glycol, glyceryl behenate, mineral oil, sodium stearyl fumarate, stearic acid, hydrogenated vegetable oil and talc.

Glidants comprises but not limited to, silicon dioxide; magnesium trisilicate, powdered cellulose, starch, talc and tribasic calcium phosphate, calcium silicate, magnesium silicate, colloidal silicon dioxide, silicon hydrogel and other materials known to one of ordinary skill in the art.

Disintegrants comprises but not limited to starches; clays; celluloses; alginates; gums; cross-linked polymers, e.g., cross-linked polyvinyl pyrrolidone or crospovidone, e.g., POLYPLASDONE XL, cross-linked sodium carboxymethylcellulose or croscarmellose sodium, e.g., AC-DI-SOL from FMC; and cross-linked calcium carboxymethylcellulose; soy polysaccharides; and guar gum. Use of disintegrant according to the present invention facilitates in the release of drug in the latter stage and thereby completely releasing the drug from the dosage form.

The pharmaceutical composition may optionally contain a surface-active agent. The preferred agent is copolymers composed of a central hydrophobic chain of polyoxypropylene (poly(propylene oxide)) and polyoxyethylene (poly(ethylene oxide)) that is well known as poloxamer. However, other agents may also be employed such as dioctyl sodium sulfosuccinate (DSS), triethanolamine, sodium lauryl sulphate (SLS), polyoxyethylene sorbitan and poloxalkol derivatives, quaternary ammonium salts or other pharmaceutically acceptable surface-active agents known to one ordinary skilled in the art.

In another embodiment, delayed release portion and one or more immediate release portions are optionally separated from each other by one or more layers of barrier coating wherein the barrier coating can be film coating, sugar coating, extended release coating, enteric coating, partial enteric coating or leaky enteric coating, bioadhesive coating and other coatings known in the art. These coatings may help pharmaceutical composition to release the drug at and for the required time.

The term barrier coating and barrier layer can be used interchangeably.

In another embodiment, one or more immediate release portions are optionally coated by barrier coating; wherein barrier coating comprises a hydrophilic or hydrophobic substance(s) or the combinations thereof.

The hydrophobic substance in the barrier coating is selected from Ammonio methacrylate copolymers type A and B as described in USP, methacrylic acid copolymer type A, B and C as described in USP, Polyacrylate dispersion 30% as described in pH. Eur., Polyvinyl acetate dispersion, ethylcellulose, cellulose acetate, cellulose propionate (lower, medium or higher molecular weight), cellulose acetate propionate, cellulose acetate butyrate, cellulose acetate phthalate, cellulose triacetate, poly(methyl methacrylate), poly(ethyl methacrylate), poly(butyl methacrylate), poly(isobutyl methacrylate), and poly(hexyl methacrylate). Poly(isodecyl methacrylate), poly(lauryl methacrylate), poly(phenyl methacrylate), poly(methyl acrylate), poly(isopropyl acrylate), poly(isobutyl actylate), poly(octadecyl acrylate), waxes such as beeswax, carnauba wax, microcrystalline wax, and ozokerite; fatty alcohols such as cetostearyl alcohol, stearyl alcohol; cetyl alcohol and myristyl alcohol; and fatty acid esters such as glyceryl monostearate, glycerol distearate; glycerol monooleate, acetylated monoglycerides, tristearin, tripalmitin, cetyl esters wax, glyceryl palmitostearate, glyceryl behenate, and hydrogenated castor oil.

The hydrophilic substance in the barrier coating is selected from celluloses or their salts or derivatives thereof, hydroxyethylcellulose, hydroxypropyl cellulose, hydroxypropyl methylcellulose, sodium carboxymethyl cellulose, alginic acid or their salts and derivatives thereof, carbomer (Carbopol™), polyethyleneoxide, xanthan gum, guar gum, locust bean gum, poly vinyl acetate, polyvinyl alcohol, lactose, PVA these hydrophilic polymers also act as pore forming agent.

These coating comprises one or more excipients selected from coating agents, opacifiers, taste-masking agents, fillers, polishing agents, colouring agents, antitacking agents and the like.

A pharmaceutical composition of the invention can be coated by a wide variety of methods. Suitable methods include compression coating, coating in a fluidized bed or a pan and hot melt (extrusion) coating. Such methods are well known to those skilled in the art.

A pharmaceutical composition of the invention can be formed by various methods known in the art but not limited to such as by dry granulation, wet granulation, melt granulation, direct compression, double compression, extrusion spheronization, layering and the like.

The solvent(s) used in wet granulation include all the solvents well known in the art or the mixtures thereof.

In another embodiment, a process for preparing pharmaceutical composition comprises:

a) preparing a delayed release portion comprising non-steroidal anti-inflammatory drug and one or more pharmaceutically acceptable excipients;
b) applying barrier layer comprising one or more hydrophilic or hydrophobic substances and one or more pharmaceutically acceptable excipients.
c) preparing immediate release portion comprising non-steroidal anti-inflammatory drug and one or more pharmaceutically acceptable excipients;
d) applying immediate release portion of step (c) onto barrier layer of (b) to form tablet.
e) preparing an immediate release portion proton pump inhibitor comprising dihydrate form of proton pump inhibitor and one or more pharmaceutically acceptable excipients.
f) applying the immediate release portion of proton pump inhibitor onto the tablet of step (d).

In another embodiment, a pharmaceutical composition of present invention found to be stable and bioequivalent to Vimovo® (Esomeprazole & Delayed release Naproxen Tablet) tablets, Distributed by Astrazeneca.

In one embodiment, the pharmaceutical composition can be used in pain and symptom relief with a reduced risk of developing gastrointestinal damage such as ulcers, erosions and hemorrhages.

In another embodiment, the pharmaceutical composition can be used in pain and symptom relief of arthritic conditions which includes but not limited to osteoarthritis, rheumatoid arthritis, juvenile arthritis, ankylosing spondylitis etc.

In yet another embodiment, the pharmaceutical composition can be used in treatment and prophylaxis of upper or lower gastrointestinal disorders associated with the use of Non Steroidal Antiinflanmmatory Drugs (NSAIDs).

In yet another embodiment, the pharmaceutical composition can be used in pain and symptom relief of Non-Erosive Reflux Disease, heart burn, Inflammatory Bowel Diseases, Upper Abdominal Pain, Nausea, Acid Regurgitation, Postoperative Bariatric Surgery, Chronic Pain, peptic ulcer, gastric ulcer, Lumbago etc.

The foregoing examples are illustrative embodiments of the invention and are merely exemplary. A person skilled in the art may make variations and modifications without deviating from the spirit and scope of the invention. All such modifications and variations are intended to be included within the scope of the invention.

### BRIEF DESCRIPTION OF DRAWINGS

FIG. 1 is a schematic diagram of a multilayered tablet composition. There is a NSAID portion (A) surrounded by an barrier layer (B) which delays the release of NSAID. Barrier layer is surrounded by immediate release portion of NSAID (C) which is further surrounded by barrier layer (D). Finally there is an immediate release portion of acid inhibitor (E) such as esomeprazole which is optionally surrounded by film coating (F).

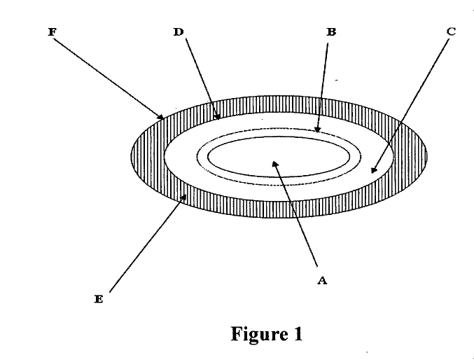

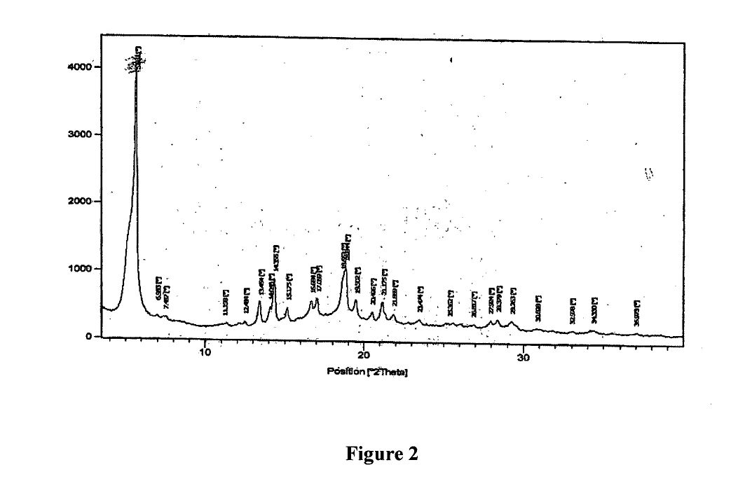

### Example 1

Ingredients

1. Delayed Release Portion
NSAID (s)
Pharmaceutically acceptable excipient(s)
Barrier Coating

2. Immediate Release Portion
NSAID (s)
Pharmaceutically acceptable excipient(s)
Barrier Coating

3. Immediate Release Portion
Acid Inhibitor
Pharmaceutically acceptable excipient(s)
Barrier Coating
1. Delayed release portion consisting of NSAID(s) and pharmaceutically acceptable excipient(s).
2. Delayed release portion is surrounded by barrier coating.
3. Immediate release portion of NSAID(s) and pharmaceutically acceptable excipient(s) is present over barrier coating of step 2
4. Barrier coating is provided over immediate release portion of step 3.
5. Immediate release portion of acid inhibitor and pharmaceutically acceptable excipient(s) is present over barrier coating of step 4.
6. Barrier coating is provided over immediate release portion of step 4

### Example 2

### Naproxen and Esomeprazole Magnesium Dihydrate Composition

### Inner Core:

| Sr. No. | Ingredients | % w/w |
|---|---|---|
| 1 | Naproxen | 65.57 |
| 2 | Povidone | 2.05 |
| 3 | Croscarmellose | 2.73 |
| | Sodium | |
| 4 | Purified Water | q.s. |

Extragranular

| | | |
|---|---|---|
| 5 | Croscarmellose | 2.73 |
| | Sodium | |
| 6 | Magnesium Stearate | 0.68 |
| 7 | Colloidal silicon | 1.37 |
| | dioxide | |

All ingredients are passed through the suitable seives.
1. Sift Naproxen, Crosscaramellose Sodium.
2. Granulate the blend of step 1 with binder solution of Povidone.
3. Dry the granules of step 2
4. Add the extragranular part to step 3 and mix well. Compress the blend using suitable tooling.
   Inner core is coated by an enteric coating,

### Enteric Coating:

| Sr. No. | Ingredients | % w/w |
|---|---|---|
| 1 | Eudragit | 2.1 |
| 2 | TEC | 0.3 |
| 3 | Talc | 0.6 |
| 4 | Isopropyl Alcohol | q.s. |
| 5 | Dichloromethane | q.s. |

1. Disperse Eudragit, TEC in Isopropyl alcohol under stirring.
2. Add Dichloromethane to step 1 under stirring.
3. Add Talc to step 2 under stirring
4. Coat the tablets with the coating solution of Step 3.
   Enteric coating layer is surrounded by a layer of NSAID

### Drug Loading of NSAID:

| Sr. No. | Ingredients | % w/w |
|---|---|---|
| 1 | Naproxen | 2.73 |
| 2 | HPMC | 2.73 |
| 3 | PEG | 0.27 |
| 4 | Isopropyl Alcohol | q.s. |
| 5 | Dichloromethane | q.s. |

1. Disperse HPMC, NSAID in Isopropyl alcohol under stirring.
2. Add Dichloromethane to step 1 under stirring.
3. Add PEG to step 2 under stirring.
4. Coat the tablets with the coating solution of Step 3.
   NSAID layer is surrounded by barrier layer

### Barrier Coating:

| Sr. No. | Ingredients | % w/w |
|---|---|---|
| 1 | HPMC | 3.81 |
| 2 | PEG | 0.38 |
| 3 | Isopropyl Alcohol | q.s. |
| 4 | Dichloromethane | q.s. |

1. Disperse HPMC in Isopropyl alcohol under stirring.
2. Add Dichloromethane to step 1 under stirring.
3. Add PEG to step 2 under stirring.
4. Coat the tablets with the coating solution of Step 3.
   Barrier coating layer is surrounded by acid inhibitor layer

### Acid Inhibitor Layer:

| Sr. No. | Ingredients | % w/w |
|---|---|---|
| 1 | Esomeprazole | 3.03 |
| | magnesium | |
| | dihydrate | |
| 2 | HPMC | 4.10 |
| 3 | Povidone | 0.68 |
| 4 | PEG | 0.41 |
| 5 | Tween | 0.1 |
| 6 | Talc | 0.68 |
| 7 | Methanol | q.s. |
| 8 | Dichloromethane | q.s. |

1. Add HPMC, esomeprazole magnesium dihydrate, Povidone, PEG, and Tween in Isopropyl alcohol under stirring.
2. Add Dichloromethane to step 1 under stirring.
3. Add Talc to step 2 under stirring.
4. Coat the tablets with the coating solution of Step 3.
   Acid inhibitor layer is surrounded by one or more barrier layer.

### X-RD Study:

The pattern X-ray diffraction for the Esomeprazole magnesium dihydrate obtained by measuring in X-ray diffractometer is shown in FIG. 2.

1. A pharmaceutical composition comprising esomeprazole magnesium, non-steroidal anti-inflammatory drug and one or more pharmaceutically acceptable excipients.

2. A pharmaceutical composition comprising esomeprazole magnesium dihydrate, non-steroidal anti-inflammatory drug and one or more pharmaceutically acceptable excipients wherein, esomeprazole magnesium dihydrate and non-steroidal anti-inflammatory drug provides co-ordinated release.

3. A pharmaceutical composition comprising esomeprazole magnesium dihydrate, non-steroidal anti-inflammatory drug and one or more pharmaceutically acceptable excipients, wherein non-steroidal anti-inflammatory drug is present in two or more portions.

4. A pharmaceutical composition comprising esomeprazole magnesium dihydrate, non-steroidal anti-inflammatory drug and one or more pharmaceutically acceptable excipients, wherein two or more portions of non-steroidal anti-inflammatory drug are separated by barrier layer.

5. The pharmaceutical composition of claim 3, wherein esomeprazole magnesium dihydrate is in immediate release portion and non-steroidal anti-inflammatory drug is in two portions, a delayed release portion and an immediate release portion.

6. The pharmaceutical composition of claim 1 is multi-layered tablet or capsule.

7. The pharmaceutical composition of claim 3, wherein non-steroidal anti-inflammatory drug present in two or more portions is same or different.

8. The pharmaceutical composition of claim 1, wherein a non-steroidal anti-inflammatory drug is selected from aspirin or naproxen.

9. A method of relieving signs and symptoms of any of osteoarthritis, rheumatoid arthritis, ankylosing spondylitis, comprising administering a pharmaceutical composition of claim 1.

10. The pharmaceutical composition of claim 1, wherein esomeprazole magnesium is present in dihydrate amorphous form.

11. A method of treating non-steroidal anti-inflammatory drug-associated gastric ulcers comprising administering a pharmaceutical composition of claim 1
